(19) United States
(12) Patent Application Publication
(10) Pub. No.: US 2020/0221944 A1
(43) Pub. Date: Jul. 16, 2020

(54) VIRTUAL REALITY-BASED OPHTHALMIC INSPECTION SYSTEM AND INSPECTION METHOD THEREOF

(71) Applicants: Ho-Fang SHEN, Taipei City (TW); Ming-Fu CHEN, Taipei City (TW)

(72) Inventors: Ho-Fang SHEN, Taipei City (TW); Ming-Fu CHEN, Taipei City (TW)

(21) Appl. No.: 16/831,445

(22) Filed: Mar. 26, 2020

Related U.S. Application Data
(63) Continuation-in-part of application No. 16/000,668, filed on Jun. 5, 2018, now Pat. No. 10,624,536.

(30) Foreign Application Priority Data
Jun. 6, 2017 (TW) 106118619

(51) Int. Cl. A61B 3/032; A61B 3/00; A61B 3/11

(52) U.S. Cl. CPC A61B 3/032; A61B 3/005; A61B 3/0025; A61B 3/0033; A61B 3/112

(57) ABSTRACT

A virtual reality-based ophthalmic inspection system includes a wearable unit, an electronic unit, and at least one detector. The wearable unit is available for an inspected object to wear the wearable unit on head. The electronic unit is assembled with the wearable unit and has a left-eye display zone and a right-eye display zone. A first visual acuity of the inspected object is identified according to a first eyesight information. The electronic unit performs a visual correction confirmation process, the at least one detector detects a condition value of corresponding the right eye and/or the left eye, and the electronic unit answers a first comparison result after comparing the condition value with a threshold value.

### CROSS REFERENCE TO RELATED APPLICATION

This application is a Continuation-in-Part of co-pending application Ser. No. 16/000,668, filed on Jun. 5, 2018, which claims priority to Taiwanese Patent Application No. 106118619, filed on Jun. 6, 2017. The entire disclosure of which is incorporated by reference herein.

### BACKGROUND

### Technical Field

The present disclosure relates to an optometry system and an optometry method and, more particularly, to a virtual reality-based ophthalmic inspection system and inspection method thereof.

### Description of Related Art

An eye chart (or called acuity chart, optotype) is used for measuring the ability of a visual system to recognize the fine structure of the object (or the spatial resolution of the visual system). It represents the most common and useful test for inspecting visual function; it may further be used to determine the lens correction associated with ocular defects. In general, the viewing distance of six meters is called far acuity, and the apparatus is called far acuity chart. Hence, six meters (or 20 feet) are considered optical infinity when person's viewing distance is approximately 6 meters at rest condition.

The visual acuity of the inspected object is identified by the smallest optotype on the eye chart seen by the inspected objected away from the eye chart with a predetermined distance, quantified optotype size, and quantified illuminant condition. The optotypes are usually letters, numbers, or geometric symbols.

However, the inspection mentioned above requires substantial space and the inspected object cannot perform the inspection by himself/herself.

### SUMMARY

One innovative aspect of the subject matter in this specification can be embodied in a virtual reality-based ophthalmic inspection system. The virtual reality-based ophthalmic inspection system comprises: a wearable unit available for an inspected object to wear the wearable unit on a head of the inspected object; an electronic unit assembled with the wearable unit and having a left-eye display zone and a right-eye display zone; and at least one detector disposed on the electronic unit. During a visual acuity inspection and a right eye of the inspected object and a left eye of the inspected object are independently inspected, a sight-target with at least one first distinguishing feature is shown on one of the left-eye display zone and the right-eye display zone, the left-eye display zone displays the sight-target while the right-eye display zone is filled with black, the right-eye display zone displays the sight-target while the left-eye display zone is filled with black, and a first size of the sight-target is successively increased before the at least one detector captures a predetermined indication. After the at least one detector received the predetermined indication, the at least one first distinguishing feature of the sight-target is changed step by step while the first size of the sight-target is fixed at a first specific size, the at least one detector further captures the at least one first distinguishing feature to identify a first eyesight information which relates to the at least one first distinguishing feature of the sight-target successively displayed on the left-eye display zone or the right-eye display zone, and a first visual acuity of the inspected object is identified according to the first eyesight information. After the first visual acuity of the inspected object is identified according to the first eyesight information, the electronic unit performs a visual correction confirmation process, the at least one detector detects a condition value of corresponding the right eye and/or the left eye, and the electronic unit answers a first comparison result after comparing the condition value with a threshold value.

In an embodiment of the present disclosure, when the electronic unit performs the visual correction confirmation process, the left-eye display zone and/or the right-eye display zone are/is flashed; the condition value is a blink number.

In an embodiment of the present disclosure, the electronic unit answers the first comparison result as success if the blink number is greater than 30.

In an embodiment of the present disclosure, the condition value is a size of a pupil.

In an embodiment of the present disclosure, the electronic unit answers the first comparison result as success if the size of the pupil is greater than 4 millimeters.

In an embodiment of the present disclosure, the virtual reality-based ophthalmic inspection system further comprises: an external display unit wirelessly connected to the electronic unit, wherein the external display unit displays same contents of the electronic unit through wireless projections.

In an embodiment of the present disclosure, after the first visual acuity of the inspected object is identified according to the first eyesight information, the electronic unit performs a comparing visual acuity inspection; during the comparing visual acuity inspection and the right eye and the left eye are independently inspected, a number with at least one second distinguishing feature is shown on one of the left-eye display zone and the right-eye display zone, the left-eye display zone displays the number while the right-eye display zone is filled with black, the right-eye display zone displays the number while the left-eye display zone is filled with black, and a second size of the number is successively increased before the at least one detector captures a voice response. After the at least one detector received the voice response, the at least one second distinguishing feature of the number is changed step by step while the second size of the number is fixed at a second specific size, the at least one detector further captures the at least one second distinguishing feature to identify a second eyesight information which relates to the at least one second distinguishing feature of the number successively displayed on the left-eye display zone or the right-eye display zone, a second visual acuity of the inspected object is identified according to the second eyesight information, and the electronic unit answers a second comparison result after comparing the second visual acuity with the first visual acuity.

One innovative aspect of the subject matter in this specification can be embodied in an ophthalmic examination method. The ophthalmic examination method includes providing a display unit comprising a left-eye display zone and a right-eye display zone; displaying a sight-target on one of the left-eye display zone and the right-eye display zone, wherein the sight-target has at least one first distinguishing feature and a first size; successively increasing the first size of the sight-target while the at least one first distinguishing feature of the sight-target is fixed; capturing a predetermined indication; and successively changing the at least one first distinguishing feature of the sight-target while the first size of the sight-target is fixed at a first specific size and capturing a first eyesight information relating to the at least one first distinguishing feature. During a visual acuity inspection and a right eye of an inspected object and a left eye of the inspected object are independently inspected, the first size of the sight-target is successively increased before at least one detector captures the predetermined indication. After the at least one detector received the predetermined indication, the at least one first distinguishing feature of the sight-target is changed step by step while the first size of the sight-target is fixed at the first specific size, the at least one detector further captures the at least one first distinguishing feature to identify the first eyesight information which relates to the at least one first distinguishing feature of the sight-target successively displayed on the left-eye display zone or the right-eye display zone, and a first visual acuity of the inspected object is identified according to the first eyesight information. After the first visual acuity of the inspected object is identified according to the first eyesight information, a visual correction confirmation process is performed, the at least one detector detects a condition value of corresponding the right eye and/or the left eye, and a first comparison result is answered after comparing the condition value with a threshold value.

In an embodiment of the present disclosure, when the visual correction confirmation process is performed, the left-eye display zone and/or the right-eye display zone are/is flashed; the condition value is a blink number.

In an embodiment of the present disclosure, the first comparison result is answered as success if the blink number is greater than 30.

In an embodiment of the present disclosure, the condition value is a size of a pupil.

In an embodiment of the present disclosure, the first comparison result is answered as success if the size of the pupil is greater than 4 millimeters.

In an embodiment of the present disclosure, an external display unit displays same contents of the display unit through wireless projections.

In an embodiment of the present disclosure, after the first visual acuity of the inspected object is identified according to the first eyesight information, a comparing visual acuity inspection is performed; during the comparing visual acuity inspection and the right eye and the left eye are independently inspected, a number with at least one second distinguishing feature is shown on one of the left-eye display zone and the right-eye display zone, the left-eye display zone displays the number while the right-eye display zone is filled with black, the right-eye display zone displays the number while the left-eye display zone is filled with black, and a second size of the number is successively increased before the at least one detector captures a voice response. After the at least one detector received the voice response, the at least one second distinguishing feature of the number is changed step by step while the second size of the number is fixed at a second specific size, the at least one detector further captures the at least one second distinguishing feature to identify a second eyesight information which relates to the at least one second distinguishing feature of the number successively displayed on the left-eye display zone or the right-eye display zone, a second visual acuity of the inspected object is identified according to the second eyesight information, and a second comparison result is answered after comparing the second visual acuity with the first visual acuity.

In an embodiment of the present disclosure, after the first visual acuity of the inspected object is identified according to the first eyesight information, a mydriatic which is suitable for the condition value is answered.

### BRIEF DESCRIPTION OF DRAWING

The present disclosure can be more fully understood by reading the following detailed description of the embodiment, with reference made to the accompanying drawings as follows:

FIG. 1 depicts a schematic diagram illustrating a virtual reality-based ophthalmic inspection system in accordance with the present disclosure;

FIG. 2 depicts a schematic diagram illustrating the operation of the virtual reality-based ophthalmic inspection system in accordance with the present disclosure;

FIGS. 3-5 depict schematic diagrams illustrating eye sights shown on a display for inspecting visual acuity of left eye;

FIGS. 6-7 depict schematic diagrams illustrating eye sights shown on the display for inspecting visual acuity of right eye;

FIG. 8 depicts the principle for inspecting the visual acuity;

FIGS. 9A-9B depict system block diagrams illustrating the virtual reality-based ophthalmic inspection system in accordance with the present disclosure;

FIG. 10 depicts schematic diagrams illustrating eye sights shown on the display for inspecting visual sensitivity of left eye;

FIG. 11 depicts schematic diagrams illustrating the Amsler grid for inspecting macular degeneration of left eye;

FIG. 12 depicts schematic diagrams illustrating the Amsler grid viewed by the inspected object having macular degeneration;

FIG. 13 depicts a flow chart of the ophthalmic inspection method in accordance with the present disclosure;

FIG. 14 depicts a schematic diagram illustrating a virtual reality-based ophthalmic inspection system in accordance with the present disclosure;

FIG. 15 depicts another schematic diagram illustrating eye sights shown on the display for inspecting visual acuity of left eye; and

FIG. 16 depicts another schematic diagram illustrating eye sights shown on the display for inspecting visual acuity of right eye.

### DETAILED DESCRIPTION

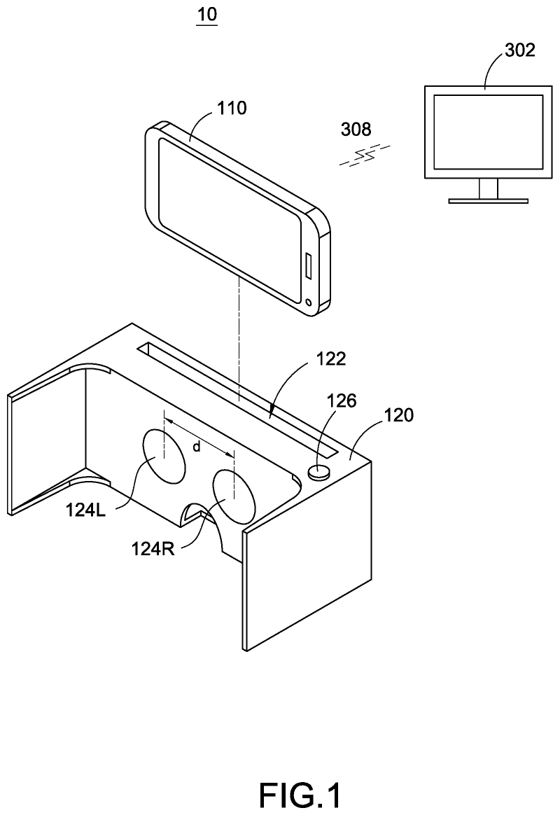

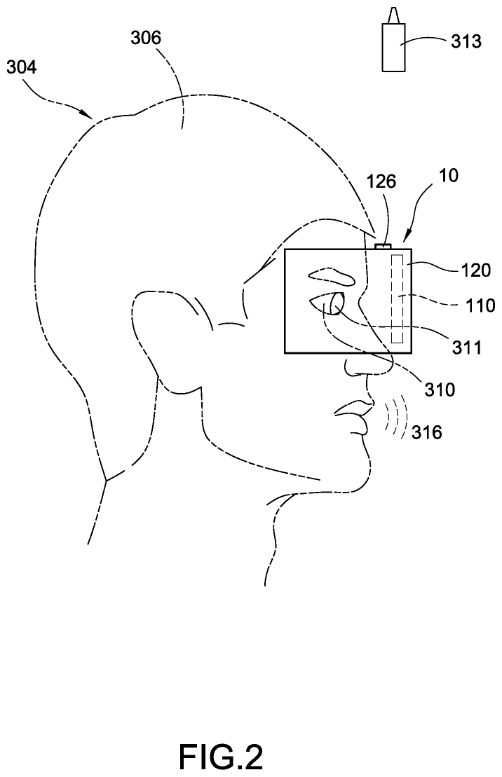

FIG. 1 is a schematic view of a virtual reality-based ophthalmic inspection system in accordance with the present disclosure, and FIG. 2 is a schematic view of the operation of the virtual reality-based ophthalmic inspection system in accordance with the present disclosure. The virtual reality-based ophthalmic inspection system 10 includes an electronic unit 110 and a wearable unit 120, and the electronic unit 110 is affixed to the wearable unit 120. The virtual reality-based ophthalmic inspection system 10 is wearable on a user's head 306, and the electronic device 110 corresponding to the user's eye is configured to display images (of eye sights). The electronic unit 110 is, for example, a smartphone. In FIG. 1, the electronic unit 110 and the wearable unit 120 are separated; in other words, the electronic unit 110 is treated as a smartphone during the electronic unit 110 is not affixed to the wearable device 120. However, in the other embodiments, the electronic unit 110 and the wearable unit 120 may be integrated-formed, i.e., the electronic unit 110 and the wearable unit 120 collectively form a headset electronic unit, which cannot be detached.

In FIG. 1, a front end of the wearable unit 120 forms an installation slot 122, and the electronic unit 110 is installed in the wearable unit 120 through the installation slot 122. The wearable unit 120 further includes two lenses 124R and 124L, which may be placed at a rear end of the wearable unit 120; the focal lengths of the lenses 124R and 124L are designed for clearly imaging the light emitted from the electronic unit 110 on user's retinas. The lenses 124R and 124L may convert light emitted from the electronic unit 110 to a parallel light, and the parallel light is then transmitted to user's eyes that have normal vision. In operation, the rear end of the wearable unit 120 may attach to user's face, and user's eyes view the images shown on the electronic unit 110 through the lenses 124R and 124L.

During an eye inspecting process, the virtual reality-based ophthalmic inspection system 10 may simulate the situation in which the inspected object covers one eye when performing visual acuity inspection on the other eye; in other words, the virtual reality-based ophthalmic inspection system 10 is configured to inspect monocular visual acuity.

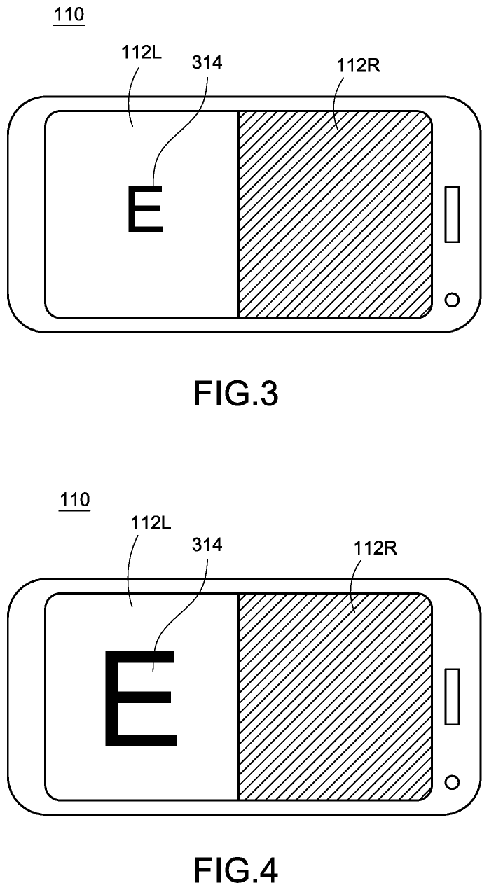

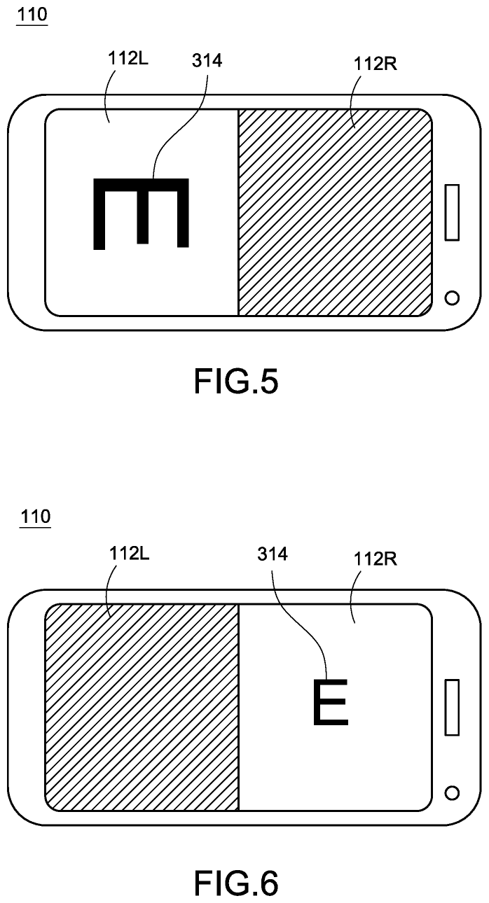

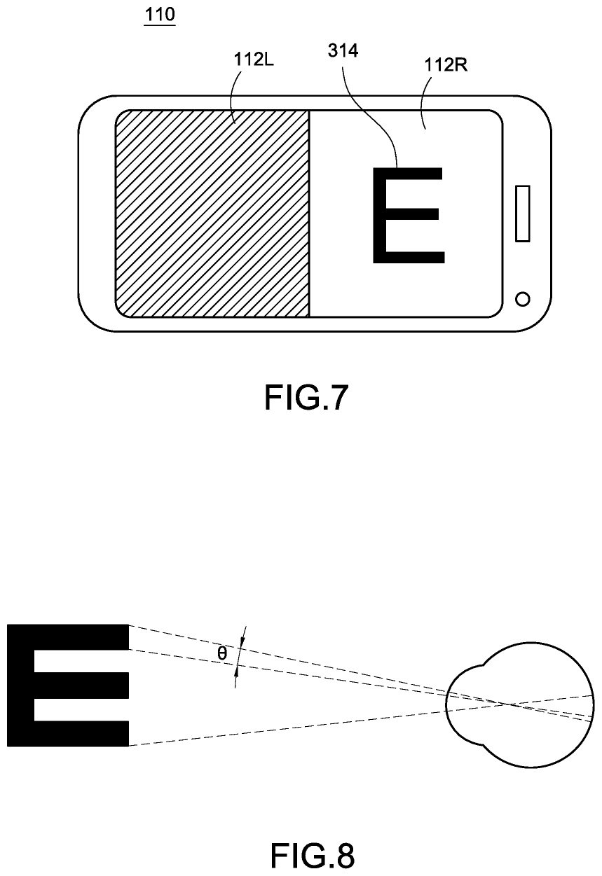

Specifically, the electronic unit 110 includes a display unit 112, which is divided into a right-eye display zone 112R for right eye 310 viewing and a left-eye display zone 112L for left eye viewing. During left eye inspecting process, the left-eye display zone 112L displays the sight-target(s) 314; the right-eye display zone 112R does not display any sight-target 314 and is filled with black (the diagonal lines showed in FIG. 3 to FIG. 5 represent the right-eye display area 112R filled with black) to simulated the situation which the inspected persons 304 covers right eye 310 when performing visual acuity inspection on the left eye 312; wherein the right-eye display zone 112R filled with black is used for completely blocking the vision of right eye 310. Similarly, during right eye inspecting process, the right-eye display zone 112R displays the sight-target(s) 314; the left-eye display zone 112L does not display any sight-target 314 and is filled with black (the diagonal lines showed in FIG. 6 to FIG. 7 represent the left-eye display area 112L filled with black) for completely blocking the vision of left eye 312. In addition, the distance between the lenses 124R and 124L is designed to prevent the image for one eye viewing from viewing by the other eye.

Visual acuity is often inspected according to the size of the sight-targets 314 viewed on display unit 112. The sight-targets 314 may be letters, numbers, images, geometric symbols or other acceptable sight-targets having a distinguish feature. The distinguish feature enables the person with the visual acuity of 1.0 to identify (the shape, the opening, or the direction) of the sight target 314. Notably, a person with visual acuity of 1.0 represents he/she can clearly identify the sight-targets 314 having a threshold visual angle of 1 minute, i.e., the minimum angle of resolution (MAR) thereof is 1 minute, as shown in FIG. 8.

In the present disclosure, the sight-target 314 is selected from a set of letters consisting essentially of capital bold character E in a Berlin Sans FB font, capital character C in a Bauhaus 93 font, and lowercase character C in a Vrindab font. The following takes the sight-target 314 “E” as an illustrated example, and the distinguish feature thereof is the orientations of the sight-target 314 “E”. As shown in Table 1, the size (or called “the point”) of the sight-target 314 corresponds to different visual acuity for eye. In Table 1, the sight-target 314 with 1 point corresponds to visual acuity of 1.2; in detail, during the inspection, if the person clearly views the sight-target 314 with 1 point on the display 112, the inspected (right or left) eye has the visual acuity of 1.2.

TABLE 1

| size of sight-target | 10 | 9 | 8 | 7 | 5 | 4 | 3 | 2 | 1 |
|---|---|---|---|---|---|---|---|---|---|
| visual acuity | 0.1 | 0.2 | 0.4 | 0.5 | 0.6 | 0.8 | 0.9 | 1.0 | 1.2 |

In an embodiment, the electronic device 110 may have a resolution of at least 2560×1440 pixels for preventing the sight-target 314 “E” from edge blurred or lines and spaced of the sight-target 314 “E” displayed unevenly. Furthermore, the illuminance of the electronic device 110 is designed to follow the standard protocols, for example, 200±120 cd/m², and the contrast between the sight-target 314 and the background of the sight-target 314 may be designed as 0.9±0.05 to enhance inspection accuracy. In the embodiment, the sight-target 314 is black, while the background is white.

Notably, in some embodiments, the brightness and color of the background may be changed according to different inspection requirement. Specifically, the brightness of the display unit 112 may directly affect the inspection accuracy; however, the display unit 112 fabricated by different foundries may have different brightness, which makes the software for performing the inspection cannot provide the same inspection result and lower the inspection accuracy. As such, a calibration procedure to adjust the brightness and color temperature of the display unit 112 when the software is activated is desired. The calibration procedure may measure the type of the electronic unit 110 or the display unit 112, and then adjust and maintain the background brightness of the display unit 112. Besides, the calibration procedure may further optimize the color temperature of the display unit 112 for reducing blue irradiation.

In the present disclosure, the size of sight-target 314 is successively increased during the visual acuity inspection, and the right eye 310 and the left eye 312 are independently inspected. In an embodiment, the virtual reality-based ophthalmic inspection system 10 may inspect the visual acuity of the left eye 312 (by the sight-target 314 shown in FIG. 3 to FIG. 5) prior to inspecting the visual acuity of the right eye 310 (by the sight-target 314 shown in FIG. 6 and FIG. 7). In the other embodiment, the virtual reality-based ophthalmic inspection system 10 may inspect the visual acuity of the right eye 310 prior to inspecting the visual acuity of the left eye 312.

In detail, the electronic device 110 may initially display the sight-target 314 with size of 1 on the display unit 112 and successively increase the size of the sight-target 314 according to Table 1. For example, the display unit 112 display the sight-target 314 shown in FIG. 3 which smaller size firstly, and then display the sight-target 314 shown in FIG. 4 until the inspected object 304 provides a predetermined indication by at least one of body movements (such as gesture, finger motions or nod) and voice to accomplish a preliminary inspection. The display period of the sight-target 314 with different size may be designed to simulate the visual effect that the sight-target 314 toward the inspected object's sight. Thereafter, an advanced inspection may be further performed to identify the visual acuity of the inspected object 304. During the advance inspection, the electronic unit 110 may display the sight-target 314 with different distinguish features while the size thereof is fixed at a specific size for several times (for example, two or three times), and performing the visual acuity inspecting procedure by the question and answering for enhancing the accuracy.

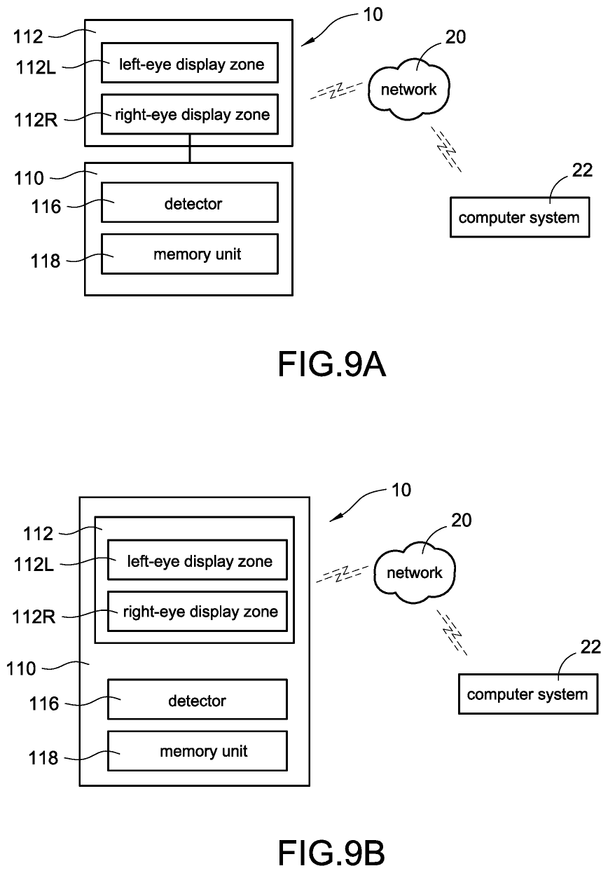

In should be noted that the virtual reality-based ophthalmic inspection system 10 may further include a detector 116 (as shown in FIG. 9A and FIG. 9B) for capturing the predetermined indication including at least one of body movements and voice. The detector 116 may be one of a button, a microphone, a camera, an ambient light sensor, a gyroscope, and a three-dimensional accelerometer, which may be the component(s) of the electronic unit 110. When the electronic unit 120 and the wearable unit 120 are separated, the wearable unit 120 may have hole(s) (not shown) at the position where the microphone, the camera, and the ambient light sensor are disposed for capturing the predetermined indication; the circuit block diagram of the system 10 which the electronic unit 120 and the wearable unit 120 are separated is shown in FIG. 9A. When the electronic unit 120 and the wearable unit 110 are integrated-formed, the detector 116 may be disposed on the wearable unit 120 and electrically connected to the electronic unit 110 as shown on FIG. 9B.

During the advance inspection, the specific size of the sight-target 314 shown on display unit 112 may be the size that displayed on the electronic unit 110 while the detector 116 captures the predetermined indication. However, the specific size mentioned above may be the size that displayed on the electronic unit 110 of a previous predetermined time period during which the predetermined indication is received so as to reduce errors produced by response time of the inspected object 304.

In addition, during the advance inspection, if the predetermined indication in relative to the distinguish feature provided by the inspected object 304 is different from the distinguish feature shown on the display unit 112, the electronic unit 110 may successively increase the size of the sight-target 314 until the predetermined indication provided by the inspected object 304 matches with the distinguish feature shown on the display unit 112 The electronic device 110 thereafter may successively display the sight-target 314 with different distinguish features while the size of the sight-target is fixed at the specific size for several times for inspecting the visual acuity of the inspected object 304 until all of the predetermined indications in relative to the distinguish features match the distinguish features successively shown on the display unit 112, and the specific sizes of the sight-targets 314 now shown in the display 112 are defined as the visual acuity the inspected object 304. Therefore, the influence of erroneous movements of the inspected object 304 on accuracy and inspection time can be effectively decreased.

After the visual acuity of left eye 312 is identified, the electronic unit 110 then displays the sight-target 314 having an initial distinguish feature and an initial size (for example, the sight-target 314 may have the size of 1 shown in Table 1) on the right-eye display zone 112R and successively increase the size of the sight-target 314 until the detector 116 captures the predetermined indication. The distinguishing feature of the sight-target 314 shown on the display 112 is then changed step by step while the size of the sight-target 314 is fixed at the specific size, and the detector 116 captures eyesight information in relation to the changed distinguishing feature until the visual acuity inspection is accomplished.

In some embodiments, the virtual reality-based ophthalmic inspection system 10 may automatically inspect the visual acuity of the inspected object 304. In some embodiments, the virtual reality-based ophthalmic inspection system 10 may be coupled to a computer system 22 through the network 20 (as shown in FIGS. 9A and 9B) and perform the visual acuity inspection according to the instruction provided by ophthalmologists or optometrists at the remote end. In some embodiments, the virtual reality-based ophthalmic inspection system 10 may store the distinguishing feature identification information within the memory unit 118 and be coupled to the computer system 22 through network 20, ophthalmologists or optometrists at remote end receive the distinguishing feature identification information and then identify the visual acuity of the inspected subject according to their eyesight information.

The virtual reality-based ophthalmic inspection system 10 of the present disclosure accomplish rapid monocular visual acuity inspection by the preliminary and advance inspection. However, in some embodiments, virtual reality-based ophthalmic inspection system 10 may further determined whether the distinguish features of the sight-targets 314 which the inspected subject are identified is the smallest size (as the size 1 shown in Table 1). As previously described, if the size of the sight-target 314 shown on the display unit 112 of the electronic unit 110 is the smallest size, then the visual acuity of the inspected objected is 1.2; however, if the size of the sight-target 314 shown on the display unit 112 of the electronic unit 110 is not the smallest size, the electronic unit 110 may reduce the size of the sight-target 314 shown on the display 112 to further inspect the visual acuity to increase accuracy.

In some embodiments, the visual acuity is identified even if the inspected object 304 cannot identify all of the distinguish feature of the sight-target 314 successively shown on the display unit 110 of the electronic unit 110. In such situation, the visual acuity of the inspected object 304 is identified if the amount of the distinguish features of the sight-targets 314 successively shown on the display 112 which are correctly recognized is greater than that are incorrectly recognized. On the contrary, of the amount of the distinguish features of the sight-targets 314 successively shown on the display 112 which are correctly recognized is less than that are incorrectly recognized, that electronic unit 110 then increases the size of the sight-target 314 until the amount of the distinguish features of the sight-targets which are correctly recognized is greater than that are incorrectly recognized.

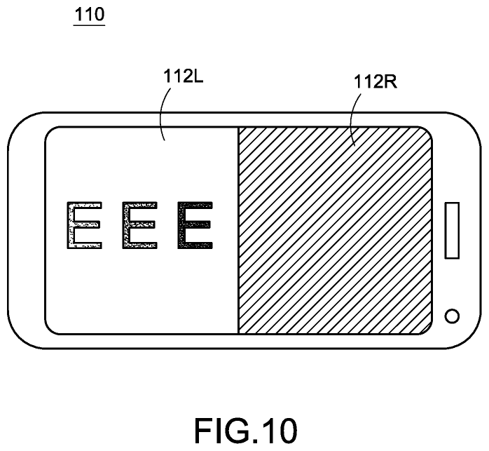

The virtual reality-based ophthalmic inspection system 10 of the present disclosure may further be configured to inspect the contrast sensitivity. Specifically, the electronic unit 110 may display a plurality of sight-targets 314 arranged in line whose contrast varies from high to low on one of the left-eye display zone 112L and the right-eye display zone 112R for inspecting visual sensitivity, as shown in FIG. 10.

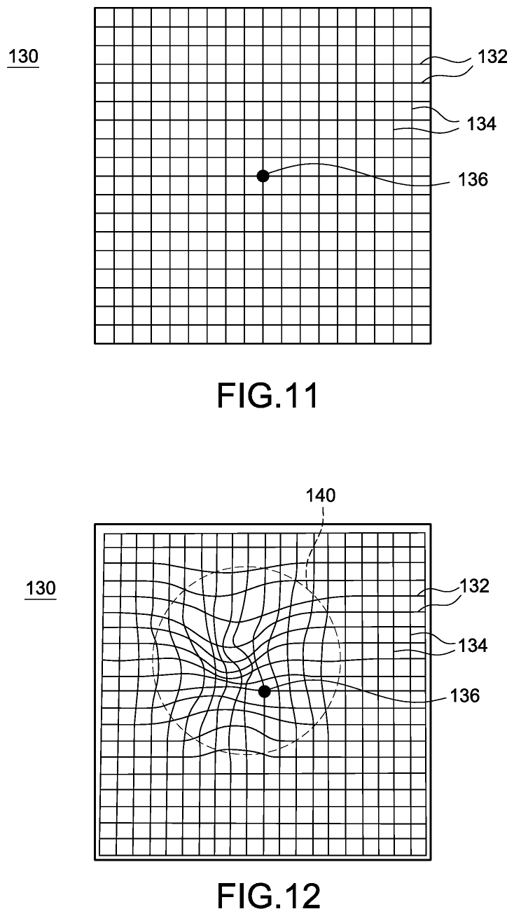

The virtual reality-based ophthalmic inspection system 10 of the present disclosure may still further be configured to inspect the macular degeneration. Specifically, the electronic unit 110 may display an Amsler grid 130 on one of the left-eye display zone 112L and the right-eye display zone 112R to the left eye 312 and the right eye 310 of the inspected object 304 to inspect the macular degeneration. FIG. 11 depicts schematic diagrams illustrating the Amsler grid 130 shown in the left-eye display zone 112L for inspecting macular degeneration of left eye 312. As can be seen in FIG. 11, the Amsler grid 130 is a square grid containing equally spaced, parallel horizontal lines 132, vertical lines 134, and a center point 136; the horizontal and vertical lines 132 and 134 collectively form a plurality of intersections, and the center point 136 is the geometric center of the Amsler grid 130.

The inspected object 304 is instructed to immediately report a preset indication (such as press button, voice indication or head rotation indication) in the severity or distribution of the distortion appear on the horizontal and vertical lines 132 and 134 of the Amsler grid 130, and the electronic unit 110 then identified that the inspected object 304 has macular degeneration. During the inspection, the electronic unit 110 may further be configured to position the portion of the Amsler grid 130 that the inspected object 304 sees as distorted; the electronic device 110 may dichotomy the left-eye display zone 112L (or the right-eye display zone 112R) by brightness to acquire the portion of the Amsler grid 130 that the inspected object 304 sees as distorted or unseen. In detail, if the distorted area is seen at the left portion of the left-eye display zone 112L, as shown in FIG. 12, then the electronic unit 110 may receive the preset indication (such as the inspected object's head 306 turns left or a button pressed) to lower the brightness of the right portion of left-eye display zone 112L. Thereafter, if the distorted area is seen at the left-upper portion of the left-eye display zone 112L, then the electronic unit 110 may receive another preset indication (such as the inspected object's head 306 lifts up or a button pressed) to lower the brightness of the lower portion of the left-eye display zone 112L. By applying the method continuously, the precisely position of the portion of the Amsler grid 130 that the inspected object 304 sees as distorted is identified.

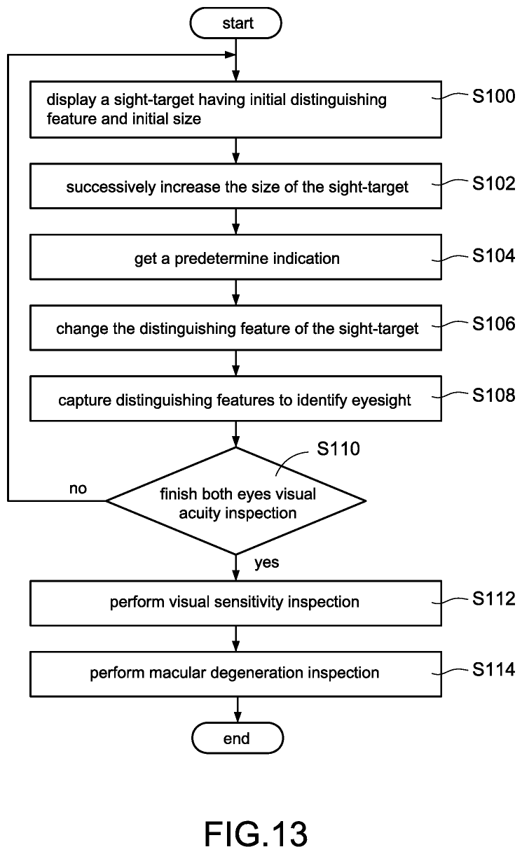

FIG. 13 depicts a flow chart of the ophthalmic inspection method in accordance with the present disclosure. The ophthalmic inspection method includes the following. First, provide a display unit 112 having a left-eye display zone 112L and a right-eye display zone 112R. Then, adjust the brightness of the left-eye display zone 112L and the right-eye display zone 112R and display a sight-target 314 with initial distinguish feature and initial size on one of the left-eye display zone 112L and the right-eye display zone 112R (step S100), wherein the initial size of the sight-target 314 is in relation to the visual acuity of 1.2 listed on Table 1. The illuminance of the left-eye display zone 112L or the right-eye display zone 112R displaying the sight-target 314 is designed to follow the standard protocols, for example, 200±120 cd/m², and the contrast between the sight-target 314 and the background of the sight-target 314 may be designed as 0.9±0.05 to enhance inspection accuracy. The left-eye display zone 112L or the right-eye display zone 112R does not display the sight-target 314, which is filled with black for completely blocking the vision of left eye 312 or right eye 310.

Then, successively increase the size of the sight-target 314 while the distinguish feature is fixed as the initial distinguish feature (step S102) until capturing a predetermined indication (step S104) by at least one of body movements and voice.

Then, change the distinguishing features of the sight-targets 314 step by step while the size of the sight-target 314 is fixed (step S106) and capture eyesight information in relation to the distinguishing features (step S108). The specific size is the size that displayed on the display unit 112 while the system 10 captures the predetermined indication, i.e., the size mentioned in the step S104.

Then, determine whether both eyes visual acuity inspection is finished (step S110); if being finished, inspect contract sensitivity of the left eye 312 and the right eye 310 (step S112) and display the inspecting result (including the visual acuity and/or the contrast sensitivity) on the displaying unit 112 or store the inspecting result in the memory unit 116, wherein the inspecting result stored in the memory unit 116 may be provided to ophthalmologists or optometrists at remote end for identifying the visual acuity and/or contrast sensitivity of the inspected subject 304. On the other hand, if both eyes visual acuity inspection is not finished, then display the sight-target 314 on the left-eye display zone 112L or the right-eye display zone 112R (step S100) and successively performs the step S102 to S108 until both eyes inspection are accomplished.

When performing the contrast sensitivity inspection, a plurality of sight-target 314 arranged along a predetermined direction are shown on one of the left-eye display zone 112L or the right-eye display zone 112R. The contracts between the sight-targets 314 and the background are different while the distinguish feature and the size thereof are the same for inspecting the contrast sensitivity of the left and right independently of the inspected object 304.

Last, inspect macular degeneration of left eye 312 and right eye 310 independently (step S114). When performing macular degeneration inspection, the electronic device 110 may display an Amsler grid 130 on one of the left-eye display zone 112L and the right-eye display zone 112R, and dichotomy the left-eye display zone 112L (or the right-eye display zone 112R) by brightness to acquire the portion of the Amsler grid 130 that the inspected object 304 sees as distorted or unseen.

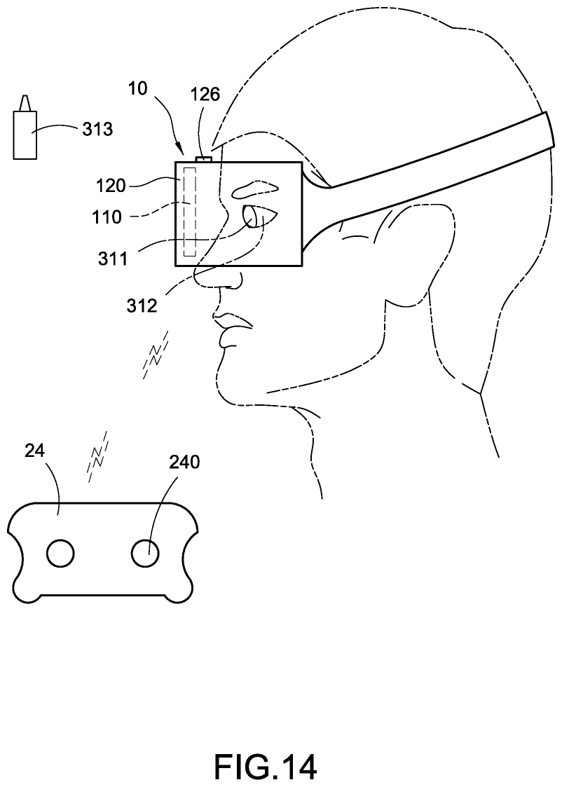

Notably, in an embodiment, the system 10 may capture the predetermined indication including at least one of body movements and voice by the detector 116 (as shown in FIG. 9A and FIG. 9b). In some embodiments, the predetermined indication may be provided by at least one button 126 disposed on the wearable unit 120 or a remote unit 20, wherein the remote unit 20 is wireless communication with the electronic unit 110, as shown in FIG. 14. Specifically, during the preliminary and advance inspection, the inspected object 304 may press the button 126 on the wearable device 120 or the button 220 on the remote unit 20 while he/she can clearly identify the distinguish features of the sight-targets 314.

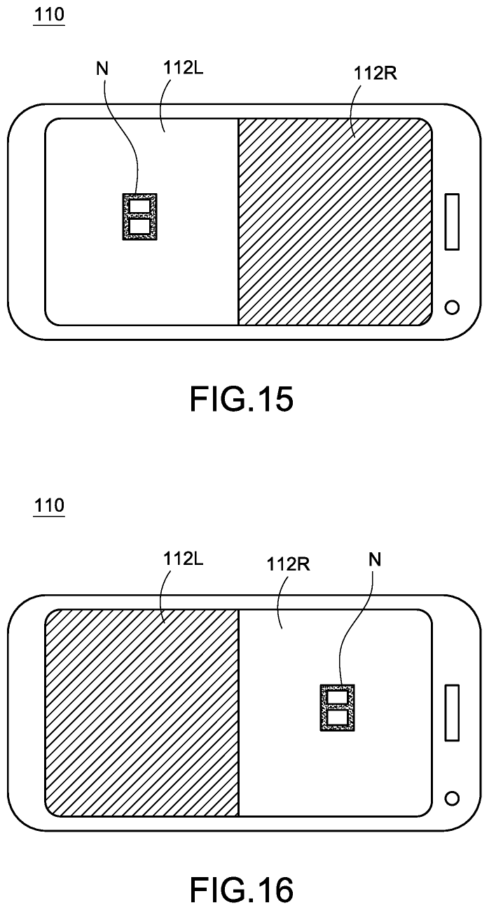

FIG. 15 depicts another schematic diagram illustrating eye sights shown on the display for inspecting visual acuity of left eye. FIG. 16 depicts another schematic diagram illustrating eye sights shown on the display for inspecting visual acuity of right eye. Please refer to FIG. 1 to FIG. 16. A virtual reality-based ophthalmic inspection system 10 comprises a wearable unit 120, an electronic unit 110, at least one detector 116 and an external display unit 302. The wearable unit 120 is available for an inspected object 304 to wear the wearable unit 120 on a head 306 of the inspected object 304. The electronic unit 110 is assembled with the wearable unit 120 and has a left-eye display zone 112L and a right-eye display zone 112R. The at least one detector 116 is disposed on the electronic unit 110. The external display unit 302 is wirelessly connected to the electronic unit 110. During a visual acuity inspection and a right eye 310 of the inspected object 304 and a left eye 312 of the inspected object 304 are independently inspected, a sight-target 314 with at least one first distinguishing feature is shown on one of the left-eye display zone 112L and the right-eye display zone 112R. The left-eye display zone 112L displays the sight-target 314 while the right-eye display zone 112R is filled with black. The right-eye display zone 112R displays the sight-target 314 while the left-eye display zone 112L is filled with black. A first size of the sight-target 314 is successively increased before the at least one detector 116 captures a predetermined indication. After the at least one detector 116 received the predetermined indication, the at least one first distinguishing feature of the sight-target 314 is changed step by step while the first size of the sight-target 314 is fixed at a first specific size. The at least one detector 116 further captures the at least one first distinguishing feature to identify a first eyesight information which relates to the at least one first distinguishing feature of the sight-target 314 successively displayed on the left-eye display zone 112L or the right-eye display zone 112R. A first visual acuity of the inspected object 304 is identified according to the first eyesight information. After the first visual acuity of the inspected object 304 is identified according to the first eyesight information, the inspected object 304 may use a mydriatic 313 onto the right eye 310 and/or the left eye 312, then the electronic unit 110 performs a visual correction confirmation process, the at least one detector 116 detects a condition value of corresponding the right eye 310 and/or the left eye 312, and the electronic unit 110 answers a first comparison result after comparing the condition value with a threshold value.

In one embodiment, when the electronic unit 110 performs the visual correction confirmation process, the left-eye display zone 112L and/or the right-eye display zone 112R are/is flashed; the condition value is a blink number; the electronic unit 110 answers the first comparison result as success if the blink number is greater than 30. In another embodiment, when the electronic unit 110 performs the visual correction confirmation process, the left-eye display zone 112L and/or the right-eye display zone 112R are/is lighting normally with backlight; the condition value is a size of a pupil 311; the electronic unit 110 answers the first comparison result as success if the size of the pupil 311 is greater than 4 millimeters, wherein the size of the pupil 311 should be between 4 millimeters to 10 millimeters if the inspected object 304 successfully uses the mydriatic 313 onto the right eye 310 and/or the left eye 312. Moreover, the external display unit 302 displays same contents of the electronic unit 110 through wireless projections 308.

After the first visual acuity of the inspected object 304 is identified according to the first eyesight information, the electronic unit 110 performs a comparing visual acuity inspection. During the comparing visual acuity inspection and the right eye 310 and the left eye 312 are independently inspected, a number N (FIG. 15 shows 8) with at least one second distinguishing feature is shown on one of the left-eye display zone 112L and the right-eye display zone 112R. The left-eye display zone 112L displays the number N while the right-eye display zone 112R is filled with black. The right-eye display zone 112R displays the number N while the left-eye display zone 112L is filled with black. A second size of the number N is successively increased before the at least one detector 116 captures a voice response 316. After the at least one detector 116 received the voice response 316, the at least one second distinguishing feature of the number N is changed step by step while the second size of the number N is fixed at a second specific size. The at least one detector 116 further captures the at least one second distinguishing feature to identify a second eyesight information which relates to the at least one second distinguishing feature of the number N successively displayed on the left-eye display zone 112L or the right-eye display zone 112R. A second visual acuity of the inspected object 304 is identified according to the second eyesight information. The electronic unit 110 answers a second comparison result after comparing the second visual acuity with the first visual acuity.

An ophthalmic examination method comprises following steps. Provide a display unit comprising a left-eye display zone and a right-eye display zone. Display a sight-target on one of the left-eye display zone and the right-eye display zone. The sight-target has at least one first distinguishing feature and a first size. Successively increase the first size of the sight-target while the at least one first distinguishing feature of the sight-target is fixed. Capture a predetermined indication. Successively change the at least one first distinguishing feature of the sight-target while the first size of the sight-target is fixed at a first specific size and capture a first eyesight information relating to the at least one first distinguishing feature. During a visual acuity inspection and a right eye of an inspected object and a left eye of the inspected object are independently inspected, the first size of the sight-target is successively increased before at least one detector captures the predetermined indication. After the at least one detector received the predetermined indication, the at least one first distinguishing feature of the sight-target is changed step by step while the first size of the sight-target is fixed at the first specific size. The at least one detector further captures the at least one first distinguishing feature to identify the first eyesight information which relates to the at least one first distinguishing feature of the sight-target successively displayed on the left-eye display zone or the right-eye display zone. A first visual acuity of the inspected object is identified according to the first eyesight information. After the first visual acuity of the inspected object is identified according to the first eyesight information, the inspected object may use a mydriatic onto the right eye and/or the left eye, then a visual correction confirmation process is performed by an electronic unit. The at least one detector detects a condition value of corresponding the right eye and/or the left eye. A first comparison result is answered after comparing the condition value with a threshold value.

In one embodiment, when the visual correction confirmation process is performed, the left-eye display zone and/or the right-eye display zone are/is flashed; the condition value is a blink number; the first comparison result is answered as success if the blink number is greater than 30. In another embodiment, when the visual correction confirmation process is performed, the left-eye display zone and/or the right-eye display zone are/is lighting normally with backlight; the condition value is a size of a pupil; the first comparison result is answered as success if the size of the pupil is greater than 4 millimeters, wherein the size of the pupil should be between 4 millimeters to 10 millimeters if the inspected object successfully uses the mydriatic onto the right eye and/or the left eye. Moreover, an external display unit displays same contents of the display unit through wireless projections. After the first visual acuity of the inspected object is identified according to the first eyesight information, the mydriatic which is suitable for the condition value is answered by the electronic unit, wherein the electronic unit has data of a plurality of the mydriatics, or data of a plurality of the mydriatics is stored in a server and is sent from the server to the electronic unit.

After the first visual acuity of the inspected object is identified according to the first eyesight information, a comparing visual acuity inspection is performed. During the comparing visual acuity inspection and the right eye and the left eye are independently inspected, a number with at least one second distinguishing feature is shown on one of the left-eye display zone and the right-eye display zone. The left-eye display zone displays the number while the right-eye display zone is filled with black. The right-eye display zone displays the number while the left-eye display zone is filled with black. A second size of the number is successively increased before the at least one detector captures a voice response. After the at least one detector received the voice response, the at least one second distinguishing feature of the number is changed step by step while the second size of the number is fixed at a second specific size. The at least one detector further captures the at least one second distinguishing feature to identify a second eyesight information which relates to the at least one second distinguishing feature of the number successively displayed on the left-eye display zone or the right-eye display zone. A second visual acuity of the inspected object is identified according to the second eyesight information. A second comparison result is answered after comparing the second visual acuity with the first visual acuity.

Although the present disclosure has been described with reference to the foregoing preferred embodiment, it will be understood that the disclosure is not limited to the details thereof. Various equivalent variations and modifications can still occur to those skilled in this art in view of the teachings of the present disclosure. Thus, all such variations and equivalent modifications are also embraced within the scope of the disclosure as defined in the appended claims.

What is claimed is:

1. A virtual reality-based ophthalmic inspection system comprising:
a wearable unit available for an inspected object to wear the wearable unit on a head of the inspected object;
an electronic unit assembled with the wearable unit and having a left-eye display zone and a right-eye display zone; and
at least one detector disposed on the electronic unit,
wherein during a visual acuity inspection and a right eye of the inspected object and a left eye of the inspected object are independently inspected, a sight-target with at least one first distinguishing feature is shown on one of the left-eye display zone and the right-eye display zone, the left-eye display zone displays the sight-target while the right-eye display zone is filled with black, the right-eye display zone displays the sight-target while the left-eye display zone is filled with black, and a first size of the sight-target is successively increased before the at least one detector captures a predetermined indication, after the at least one detector received the predetermined indication, the at least one first distinguishing feature of the sight-target is changed step by step while the first size of the sight-target is fixed at a first specific size, the at least one detector further captures the at least one first distinguishing feature to identify a first eyesight information which relates to the at least one first distinguishing feature of the sight-target successively displayed on the left-eye display zone or the right-eye display zone, and a first visual acuity of the inspected object is identified according to the first eyesight information;
wherein after the first visual acuity of the inspected object is identified according to the first eyesight information, the electronic unit performs a visual correction confirmation process, the at least one detector detects a condition value of corresponding the right eye and/or the left eye, and the electronic unit answers a first comparison result after comparing the condition value with a threshold value.

2. The virtual reality-based ophthalmic inspection system of claim 1, wherein when the electronic unit performs the visual correction confirmation process, the left-eye display zone and/or the right-eye display zone are/is flashed; the condition value is a blink number.

3. The virtual reality-based ophthalmic inspection system of claim 2, wherein the electronic unit answers the first comparison result as success if the blink number is greater than 30.

4. The virtual reality-based ophthalmic inspection system of claim 1, wherein the condition value is a size of a pupil.

5. The virtual reality-based ophthalmic inspection system of claim 4, wherein the electronic unit answers the first comparison result as success if the size of the pupil is greater than 4 millimeters.

6. The virtual reality-based ophthalmic inspection system of claim 1 further comprising:
an external display unit wirelessly connected to the electronic unit,
wherein the external display unit displays same contents of the electronic unit through wireless projections.

7. The virtual reality-based ophthalmic inspection system of claim 1, wherein after the first visual acuity of the inspected object is identified according to the first eyesight information, the electronic unit performs a comparing visual acuity inspection; during the comparing visual acuity inspection and the right eye and the left eye are independently inspected, a number with at least one second distinguishing feature is shown on one of the left-eye display zone and the right-eye display zone, the left-eye display zone displays the number while the right-eye display zone is filled with black, the right-eye display zone displays the number while the left-eye display zone is filled with black, and a second size of the number is successively increased before the at least one detector captures a voice response, after the at least one detector received the voice response, the at least one second distinguishing feature of the number is changed step by step while the second size of the number is fixed at a second specific size, the at least one detector further captures the at least one second distinguishing feature to identify a second eyesight information which relates to the at least one second distinguishing feature of the number successively displayed on the left-eye display zone or the right-eye display zone, a second visual acuity of the inspected object is identified according to the second eyesight information, and the electronic unit answers a second comparison result after comparing the second visual acuity with the first visual acuity.

8. An ophthalmic examination method comprising:
providing a display unit comprising a left-eye display zone and a right-eye display zone;
displaying a sight-target on one of the left-eye display zone and the right-eye display zone, wherein the sight-target has at least one first distinguishing feature and a first size;
successively increasing the first size of the sight-target while the at least one first distinguishing feature of the sight-target is fixed;
capturing a predetermined indication; and
successively changing the at least one first distinguishing feature of the sight-target while the first size of the sight-target is fixed at a first specific size and capturing a first eyesight information relating to the at least one first distinguishing feature,
wherein during a visual acuity inspection and a right eye of an inspected object and a left eye of the inspected object are independently inspected, the first size of the sight-target is successively increased before at least one detector captures the predetermined indication, after the at least one detector received the predetermined indication, the at least one first distinguishing feature of the sight-target is changed step by step while the first size of the sight-target is fixed at the first specific size, the at least one detector further captures the at least one first distinguishing feature to identify the first eyesight information which relates to the at least one first distinguishing feature of the sight-target successively displayed on the left-eye display zone or the right-eye display zone, and a first visual acuity of the inspected object is identified according to the first eyesight information;
wherein after the first visual acuity of the inspected object is identified according to the first eyesight information, a visual correction confirmation process is performed, the at least one detector detects a condition value of corresponding the right eye and/or the left eye, and a first comparison result is answered after comparing the condition value with a threshold value.

9. The ophthalmic examination method of claim 8, wherein when the visual correction confirmation process is performed, the left-eye display zone and/or the right-eye display zone are/is flashed; the condition value is a blink number.

10. The ophthalmic examination method of claim 9, wherein the first comparison result is answered as success if the blink number is greater than 30.

11. The ophthalmic examination method of claim 8, wherein the condition value is a size of a pupil.

12. The ophthalmic examination method of claim 11, wherein the first comparison result is answered as success if the size of the pupil is greater than 4 millimeters.

13. The ophthalmic examination method of claim 8, wherein an external display unit displays same contents of the display unit through wireless projections.

14. The ophthalmic examination method of claim 8, wherein after the first visual acuity of the inspected object is identified according to the first eyesight information, a comparing visual acuity inspection is performed; during the comparing visual acuity inspection and the right eye and the left eye are independently inspected, a number with at least one second distinguishing feature is shown on one of the left-eye display zone and the right-eye display zone, the left-eye display zone displays the number while the right-eye display zone is filled with black, the right-eye display zone displays the number while the left-eye display zone is filled with black, and a second size of the number is successively increased before the at least one detector captures a voice response, after the at least one detector received the voice response, the at least one second distinguishing feature of the number is changed step by step while the second size of the number is fixed at a second specific size, the at least one detector further captures the at least one second distinguishing feature to identify a second eyesight information which relates to the at least one second distinguishing feature of the number successively displayed on the left-eye display zone or the right-eye display zone, a second visual acuity of the inspected object is identified according to the second eyesight information, and a second comparison result is answered after comparing the second visual acuity with the first visual acuity.

15. The ophthalmic examination method of claim 8, wherein after the first visual acuity of the inspected object is identified according to the first eyesight information, a mydriatic which is suitable for the condition value is answered.
